(19) United States
(12) Patent Application Publication
(10) Pub. No.: US 2026/0221292 A1
(43) Pub. Date: Jul. 30, 2026

(54) METHODS, SYSTEMS, AND RELATED ASPECTS FOR THE AUTOMATED OPTIMIZATION AND INDIVIDUALIZATION OF CLINICAL MANAGEMENT

(71) Applicant: THE JOHNS HOPKINS UNIVERSITY, Baltimore, MD (US)

(72) Inventors: Cedric MANLHIOT, Baltimore, MD (US); Shelby KUTTY, Baltimore, MD (US); Bhargava Kumar CHINNI, Baltimore, MD (US); Jason Andrew HEARN, Baltimore, MD (US)

(21) Appl. No.: 19/148,758

(22) PCT Filed: Jan. 10, 2024
(86) PCT No.: PCT/US2024/010994

§ 371 Date: Jul. 17, 2025

Related U.S. Application Data
(60) Provisional application No. 63/480,358, filed on Jan. 18, 2023.

(51) Int. Cl. G16H 50/70

(52) U.S. Cl. CPC G16H 50/70

(57) ABSTRACT

Examples may provide computer-implemented method of predicting and optimizing a medical outcome of a patient. The method includes identifying relationships among a set of characteristics of an initial patient population, a set of therapies, and a set of outcomes in a given clinical scenario to produce a set of identified relationships. The method also includes identifying modifiable levers that can be optimized in the set of identified relationships to produce a set of identified modifiable levers. In addition, the method also includes adjusting the set of identified modifiable levers applicable to a selected patient population to produce a reduction in a predicted probability of a negative outcome for the patient. Additional methods and related systems and computer readable media are also provided.

### CROSS REFERENCE TO RELATED APPLICATIONS

This application is the national stage entry of International Patent Application No. PCT/US2024/010994, filed on Jan. 10, 2024, and published as WO 2024/155490 A1 on Jul. 25, 2024, which claims the benefit of U.S. Provisional Patent Application No. 63/480,358, filed Jan. 18, 2023, which are hereby incorporated by reference herein in their entireties.

### FIELD

This disclosure relates generally to machine learning algorithms, e.g., in the context of medical applications, such as therapy selection and clinical trial design.

### BACKGROUND

There is a complex interrelation among the various clinical risk factors, therapy, and outcomes in patients in many different clinical scenarios. Traditional risk prediction models, generally based on regression analysis, have inherent limits in these types of scenarios, first because they can only consider a single outcome at a time, thus ignoring situations where outcomes influence each other. Second, outside of pre-defined interactions, they are unable to consider how independent variables influence each other. Finally, only a limited number of factors can be included in such models, which is particularly challenging for many clinical scenarios and often involve the use of data reduction methods to combine risk factors, obfuscating important relationships and the presence of modifiable risk factors that could be targets for interventions.

Accordingly, it is apparent that there is a need for additional modelling methods of determining relationships among clinical risk factors, therapy, and outcomes in patients in various clinical scenarios.

The development of complex clinical risk modelling methods has not been successfully automated across a variety of clinical scenarios and patient populations.

Clinical risk models have not previously been used to automatically optimize patient management over multiple modifiable clinical factors or therapies.

### SUMMARY

The present disclosure provides, in certain aspects, an artificial intelligence (AI) system capable of significantly expanding the reach and utility of prediction models for patient care and research by removing the core limitation of prediction models, namely, that they must be used strictly under the conditions in which they were created. This opens the door to many applications for the systems and related aspects disclosed herein. For example, the algorithms of the present disclosure can be used to automatically develop a virtually infinite number of optimization algorithms and to optimize any clinical trial. These and other aspects will be apparent upon a complete review of the present disclosure, including the accompanying figures.

The present disclosure provides various methods of generating and/or applying prediction models for patient care and/or research. In some embodiments, for example, the methods disclosed herein comprise performing directed and predicted data transformations to resolve direct relationships between similar variables that are expressed differently between different data sources. In some embodiments, the methods disclosed herein comprise predicting missing data elements in a given data set. In some embodiments, the methods disclosed herein comprise predicting the missing data elements using a variable-specific self-tuning, single-layer neural network. In some embodiments, the methods disclosed herein comprise predicting missing variables for a given patient in the initial patient population and/or the selected patient population based at least in part on other data present in the given data set to produce a patient-level prediction for the given patient. In some embodiments, the methods disclosed herein comprise predicting the missing data elements one variable at a time in an ascending order of a proportion of missing data points.

In some embodiments, the methods disclosed herein comprise selecting k patients from the initial patient population based upon one or more parameters, wherein k comprises one or more patients. The parameters are selected by an end-user and/or by a computer based on a similarity measure to a target population. In some embodiments, the methods disclosed herein comprise determining the similarity measure using an unsupervised machine learning algorithm. An end-user determines k based at least in part on an expected degree of algorithm performance. The expected degree of algorithm performance comprises an accuracy measure of the algorithm and a prediction interval around a given prediction. In some embodiments, the methods disclosed herein comprise identifying the relationships among a set of characteristics of an initial patient population, a set of therapies, and extracting the data necessary to mathematically quantify those relationships from a common data pool.

According to various embodiments, a method of generating a graph model is presented. The method comprises reducing the graph model by a clustering of variables and by identifying a minimum spanning tree connecting specific risk factors or interventions to a given outcome. The method comprises identifying the modifiable levers by identifying modifiable risk factors in component variables in clusters connected to an outcome of interest. The method includes developing a neural network constrained by the graph model to predict the risk of outcome(s) in a given clinical scenario from identified relationships among sets of clinical data, community-based data, and molecular marker data. A computer-implemented method of predicting and optimizing a medical outcome of a patient is presented. The method includes identifying one or more relationships among a set of characteristics of an initial patient population, a set of therapies, and a set of outcomes in a given clinical scenario to produce a set of identified relationships. The neural network is able to calculate the probability of the outcome(s) for a specific patient given the clinical data, community-based data, and molecular marker data associated with that specific patient.

According to various embodiments, the methods disclosed herein also include identifying one or more modifiable levers that can be optimized in the set of identified relationships to produce a set of identified modifiable levers. In some embodiments, the modifiable levers comprise clinical factors, medications, risk factors that influence outcomes, and/or interventions associated with a probability of a negative outcome.

Various additional optional features of the above embodiments include the following. The method comprises simulating an effect of lever optimization on downstream variables and outcomes by (a) organizing one or more features from the minimum spanning tree sequentially based on biological and/or temporal ordering and predicting one or more variables that are downstream of the modifiable levers, and (b) predicting a probability of a prespecified outcome for a given patient in the initial patient population and/or the selected patient population using initial or current values of the modifiable levers to produce one or more benchmark values that are used in adjusting the set of identified modifiable levers applicable to the selected patient population.

Various additional optional features of the above embodiments also include the following. The method comprises determining an optimal value of one or more of the levers in the set of identified modifiable levers for a given patient. The method comprises producing an optimization grid for the given patient, wherein the levers in the set of identified modifiable levers are allowed to vary based on one or more pre-defined ranges and steps. The optimization grid comprises substantially all combinations of the levers in the set of identified modifiable levers. The method comprises producing a prediction of the probability of outcome for each row in the optimization grid. The method comprises using a row in the optimization grid with a lowest predicted probability of outcome to manage care for the given patient. The method comprises using the optimal value of the one or more of the levers to manage care for the given patient. The method comprises adjusting one or more lever values to assess an effect and/or to predict one or more outcomes of a clinical trial. The method comprises adjusting the initial patient population and/or the selected patient population based at least in part on identified patient groups most and/or least likely to favorably respond to a given treatment using the optimization grid. The method comprises using an entire cluster as a modifiable lever to produce an optimized cluster. The method comprises using the optimized cluster to identify one or more targets suitable for a given therapeutic administration.

In some embodiments, the methods of the present disclosure involve the use of a set of neural networks, one for each lever, that are trained to predict the optimal values for levers for a given patient without having to use the grid mechanism.

Various optional features of the above embodiments include the following. The medical outcome comprises a therapeutic outcome. The modifiable levers comprise clinical factors, medications, risk factors that influence outcomes, and/or interventions associated with a probability of a negative outcome. The given scenario can comprise any clinical scenario which can be reasonably described by the available underlying data. In some embodiments, for example, the given scenario comprises thrombohemorrhagic complications of pediatric cardiac bypass, hypoxic ischemic encephalopathy (HIE), or a response to therapeutic hypothermia (TH). The method further comprises determining an optimal value for each of one or more levers in a set of optimized levers for the patient to produce a set of optimal values for the patient. The method further comprises using the set of optimal values for the patient to identify one or more therapies for the patient to produce a set of identified therapies. The method further comprises administering one or more identified therapies in the set of identified therapies to the patient. The method further comprises using one or more selected therapies as levers in the selected patient population. The selected therapies may comprise a pharmaceutical therapy or a non-pharmaceutical intervention.

Various additional optional features of the above embodiments include the following. The method further comprises predicting one or more effects, and/or one or more factors associated with a positive and negative effect, of the selected therapies to produce a set of predicted effects and/or factors. The method further comprises using the set of predicted effects and/or factors to design of a clinical trial related to at least one of the selected therapies. The clinical trial relates to a clinical scenario that comprises thrombohemorrhagic complications of pediatric cardiac bypass.

According to various embodiments, a system for generating a set of optimizing neural network is presented. The system can be used to implement of the above embodiments or those otherwise disclosed herein. The system includes a processor; and a memory communicatively coupled to the processor, the memory storing instructions which, when executed on the processor, perform operations including: performing computational simulations using the electronic neural network to identify modifiable levers in a given clinical scenario from identified relationships among sets of clinical data, community-based data, and molecular marker data to produce a set of identified modifiable levers, wherein the modifiable levers comprise clinical factors, medications, risk factors that influence outcomes, and/or interventions associated with a probability of a negative outcome, and adjusting the set of identified modifiable levers using the electronic neural network to produce a reduction in a predicted probability of a negative outcome at a level of a given patient.

According to various embodiments, a system for predicting and optimizing a medical outcome of a patient is presented. The system can be used to implement of the above embodiments or those otherwise disclosed herein. The system includes a processor; and a memory communicatively coupled to the processor, the memory storing instructions which, when executed on the processor, perform operations including: identifying one or more relationships among a set of characteristics of an initial patient population, a set of therapies, and a set of outcomes in a given clinical scenario to produce a set of identified relationships; identifying one or more modifiable levers that can be optimized in the set of identified relationships to produce a set of identified modifiable levers; and adjusting the set of identified modifiable levers applicable to a selected patient population to produce a reduction in a predicted probability of a negative outcome for the patient.

According to various embodiments, a computer readable media for generating an optimized neural network is presented. The computer readable media can be used to implement of the above embodiments or those otherwise disclosed herein. The computer readable media includes non-transitory computer executable instruction which, when executed by at least electronic processor perform at least: performing computational simulations using the electronic neural network to identify modifiable levers in a given clinical scenario from identified relationships among sets of clinical data, community-based data, and molecular marker data to produce a set of identified modifiable levers, wherein the modifiable levers comprise clinical factors, medications, risk factors that influence outcomes, and/or interventions associated with a probability of a negative outcome, and adjusting the set of identified modifiable levers using the electronic neural network to produce a reduction in a predicted probability of a negative outcome at a level of a given patient.

According to various embodiments, a computer readable media for predicting and optimizing a medical outcome of a patient is presented. The computer readable media can be used to implement of the above embodiments or those otherwise disclosed herein. The computer readable media includes non-transitory computer executable instruction which, when executed by at least electronic processor perform at least: identifying one or more relationships among a set of characteristics of an initial patient population, a set of therapies, and a set of outcomes in a given clinical scenario to produce a set of identified relationships; identifying one or more modifiable levers that can be optimized in the set of identified relationships to produce a set of identified modifiable levers; and adjusting the set of identified modifiable levers applicable to a selected patient population to produce a reduction in a predicted probability of a negative outcome for the patient.

### DRAWINGS

The above and/or other aspects and advantages will become more apparent and more readily appreciated from the following detailed description of examples, taken in conjunction with the accompanying drawings, in which:

FIG. 1A is a flow chart that schematically shows exemplary method steps of generating an optimized electronic neural network according to some aspects disclosed herein;

FIG. 1B is a flow chart that schematically shows exemplary method steps of predicting and optimizing a medical outcome of a patient according to some aspects disclosed herein;

FIG. 1C is a flow chart that schematically shows exemplary method steps according to some aspects disclosed herein;

FIG. 2 is a schematic diagram of an exemplary system suitable for use with certain aspects disclosed herein;

FIG. 3 is an example of a clustering network created with discovery data. Predicted probability of abnormal NICHD reduced by 6.2% via network optimization using clinical levers (hematocrit, platelet, blood pH). Circles represent clusters of features, size reflects node centrality, and color is risk dimension). Lines are mathematical connections between nodes; and

FIG. 4 is a flow chart that schematically shows exemplary method steps according to some aspects disclosed herein.

### DEFINITIONS

In order for the present disclosure to be more readily understood, certain terms are first defined below. Additional definitions for the following terms and other terms may be set forth throughout the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only and is not intended to be limiting. Further, unless defined otherwise, all technical and scientific terms used herein have the same meaning as commonly understood by one of ordinary skill in the art to which this disclosure pertains. In describing and claiming the methods, systems, and computer readable media, the following terminology, and grammatical variants thereof, will be used in accordance with the definitions set forth below.

Data set: As used herein, “data set” refers to a group or collection of information, values, or data points related to or associated with one or more objects, records, and/or variables. In some embodiments, a given data set is organized as, or included as part of, a matrix or tabular data structure. In some embodiments, a data set is encoded as a feature vector corresponding to a given object, record, and/or variable, such as a given test or reference subject. For example, a medical data set for a given subject can include one or more observed values of one or more variables associated with that subject.

Electronic neural network: As used herein, “electronic neural network” or simply “neural network” refers to a machine learning algorithm or model that includes layers of at least partially interconnected artificial neurons (e.g., perceptrons or nodes) organized as input and output layers with one or more intervening hidden layers that together form a network that is or can be trained to classify data, such as test subject medical data sets (e.g., medical images or the like). An electronic neural network is sometimes referred to herein as an “artificial neural network” or “ANN”.

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), multiple-instance learning (MIL), support vector machines, decision trees (e.g., recursive partitioning processes such as CART-classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Patient: As used herein, “patient” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A patient can be a healthy individual, an individual that has or is suspected of having a disease or pathology or a predisposition to the disease or pathology, or an individual that is in need of therapy or suspected of needing therapy. The terms “individual” or “subject” are intended to be interchangeable with “patient.”

Value: As used herein, “value” generally refers to an entry in a dataset that can be anything that characterizes the feature to which the value refers. This includes, without limitation, numbers, words or phrases, symbols (e.g., + or −) or degrees.

### DESCRIPTION OF THE EMBODIMENTS

Reference will now be made in detail to example implementations. These embodiments are described in sufficient detail to enable those skilled in the art to practice the invention and it is to be understood that other embodiments may be utilized and that changes may be made without departing from the scope of the invention. The following description is, therefore, merely exemplary.

### I. Introduction

There are many complex clinical scenarios for which the ability to predict and then optimize patient outcomes at an individual level could be beneficial. These include among others, the direct individualization of therapy for patient (i.e. bedside decision support systems) and the optimization of the design of clinical trials by providing an in-silico simulation of outcomes under varying conditions. Unfortunately, these kinds of prediction are currently possible only when algorithms have been specifically designed for a given purpose and in the same patient population. In many cases, particularly for complex medical conditions, these types of algorithms are not available. In these contexts, predictive algorithms are either applied inappropriately and/or treatments/trials are selected/designed based on expert opinion.

In some aspects, the present disclosure relates to a multi-algorithm software platform to address these problems. In some embodiments, the platform performs three fundamental tasks: 1) it automatically identifies the critical pathways between patient characteristics, treatment and outcomes in a given clinical scenario, 2) within those critical pathways, it identifies modifiable levers (clinical factors and medications) that can be optimized and 3) it learns to optimize those levers for a given group of patients. In some embodiments, the algorithm can then be used in one of two ways: first, the optimal value for each levers can be calculated for a specific patient to help guide therapy for this individual patient; second, a specific drug can be included as a lever in a group of patients and allow us to both predict the effect of the drug and the factors associated with a positive and negative effect of the drug, thus optimizing the design of a clinical trial of that drug. In some embodiments, the platform described herein is used for a single clinical scenario (e.g., thrombohemorrhagic complications of pediatric cardiac bypass), however the approaches can be expanded to any clinical scenario and can even encompass complete medical domains.

There are typically multiple components working together for the algorithms of the present disclosure, a technical description of each algorithm is provided below. The algorithms are presented in the order that are executed by the software in this exemplary embodiment.
1) Feature mapping algorithm: This algorithm resolves direct relationships between similar variables that are expressed differently between different sources of data. This algorithm includes both directed data transformations (where variable x can be calculated directly from y) and predicted data transformation (where variable x can be predicted directly from y). Predicted data transformation will be done using a self-tuning single layer neural network.
2) Case selection algorithm: This algorithm selects k patients from the available patients, parameters for selection can either be determined directly by the end-user, can be selected based on similarity to the target population (with similarity determined through an unsupervised machine learning algorithm) or a combination of both approaches. In this algorithm, k is determined by the degree of algorithm performance expected (overall accuracy of the model and prediction interval around the prediction) as determined by the end-user.
3) Directed prediction of missing data elements: This algorithm uses variable-specific self-tuning, single layer neural network to predict missing data elements in the data environment. Values for missing variables are predicted for each patient individually based on the rest of the available data, thus generating patient-level predictions as opposed to standard group-level imputations. Missing data points are predicted one variable at a time in ascending order of the proportion of missing data points.
4) Clustering graph model: This algorithm automatically identifies the important pathway(s) between patient characteristics, treatment and outcomes. The clustering graph model is a fully connected graph (n by n) that get reduced both through clustering of variables (using unsupervised learning) and by identifying the minimum spanning tree connecting specific risk factors or interventions to an outcome. Within each connected cluster, component variables are scanned for modifiable risk factors which can then be used as levers for optimization (end-users do not necessarily have to optimize all levers at once).
5) Propagating neural networks: This algorithm simulates the effect of lever optimization on downstream variables and outcomes. First the minimum spanning tree is converted to a clustering neural network (neural network with the first layer constrained by the structure of the minimum spanning tree). Features from the minimum spanning tree are then organized sequentially (based on biological and/or temporal ordering) where all variables downstream of the levers are predicted (again in a sequential fashion; using single-layer, self-tuning neural networks). Then the propagating neural network predicts the probability of the prespecified outcome for each individual patient with the levers at their current value; this value will become the benchmark for the optimizing grid.
6) Optimizing grid: this algorithm determines the optimal value, at the individual patient level, for each lever. Then an optimization grid is created for each patient where all levers are allowed to vary based on pre-defined range and step (based on clinical situation). The optimization grid then includes all combinations of levers/step (grid size is a factorial of the number of steps for each levers). For each row of the grid, the propagating neural network generates a prediction of the probability of outcome. The row with the lowest probability of outcomes is then deemed to represent the “optimal” management for this patient.

In some embodiments, the optimizing grid is used in three ways:
A) Individualization of patient care: Inform patient management by providing optimized lever directly to physicians. Since generating the optimizing grid is computationally intensive, an additional step can be added where neural networks are trained to optimize levers directly.
B) Optimize the design of clinical trials: Levers can be artificially manipulated to investigate their effect and predict the outcomes of future clinical trials. The underlying patient population can also be changed based on an examination of the optimizing grid to identify patient groups most and least likely to respond to a given treatment.
C) Investigate potential druggable targets: In some cases, clusters included in the minimum spanning tree contain no modifiable risk factors. However, the entire cluster can be used as a lever and thus the identification of clusters that can be optimized can also identify druggable targets.

In some exemplary embodiments, the platform is implemented using data from a previous observational study of thrombohemorrhagic complications in children undergoing cardiac surgery; in the original trial 42.1% of all patients experienced the outcome of interest. The data was used from 398 patients and 225 features to design aspects of the platform. The minimum spanning tree included 218 features in 22 clusters. We identified 8 levers from the minimum spanning tree which were used in the optimization grid and 83 features were sequentially behind at least one lever and thus had to be predicted through propagating neural networks. After optimization of the 8 levers, the expected rate of thrombohemorrhagic complication in this cohort is predicted to be 18.5%, an absolute reduction of 24%.

The software platforms and other aspects described herein greatly expand the utility of prediction models for patient care and research by removing the core limitation of prediction models—i.e. that they have to be used strictly under the conditions they were created. This opens the door to many uses. The platforms can be used to develop a virtually infinite number of optimization algorithms and to optimize any clinical trial. Some embodiments described herein are limited to cardiovascular diseases, but the processes of the present disclosure can be readily adapted for use with other disease entities.

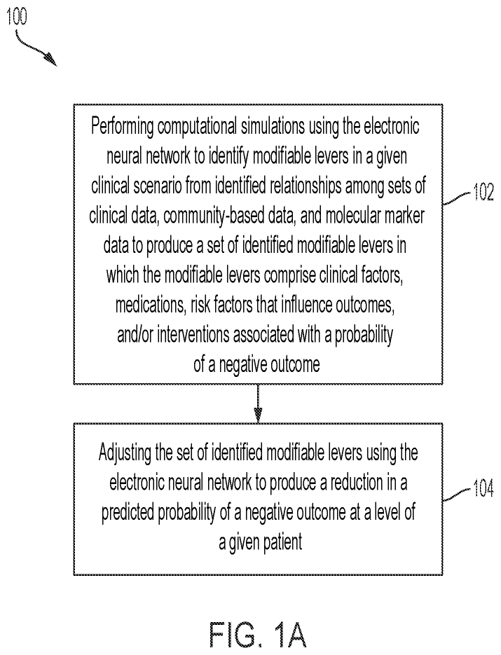

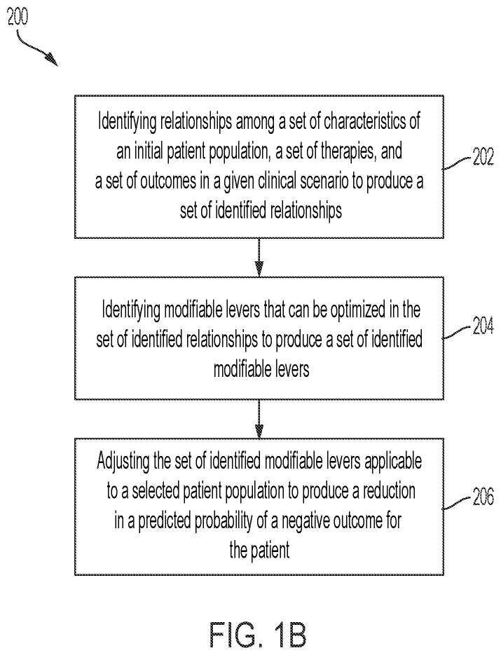

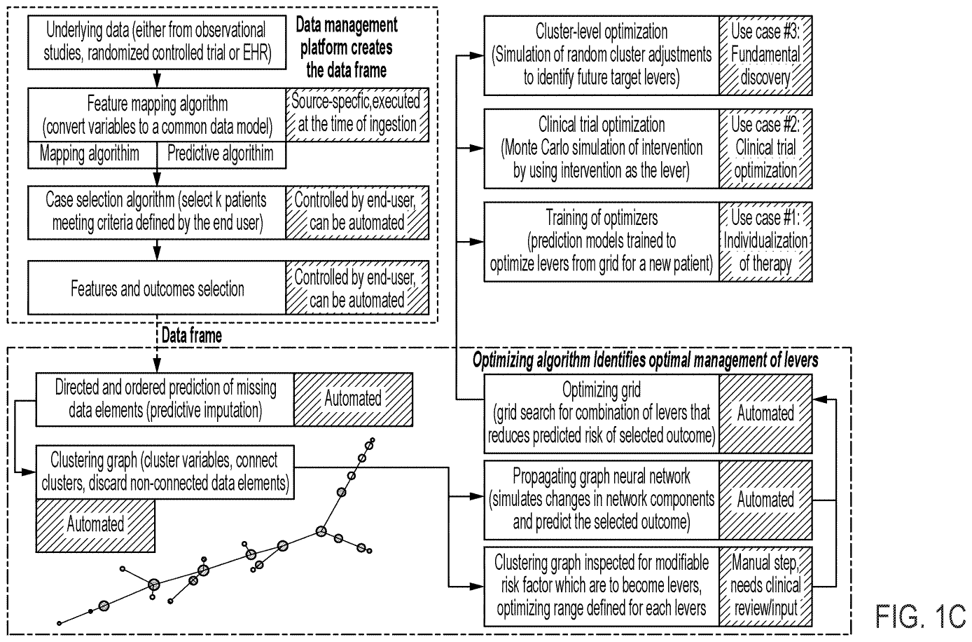

To further illustrate some aspects of the present disclosure, FIGS. 1A-1C provide flow charts that schematically shows exemplary method steps according to some embodiments. In particular, FIG. 1A is a flow chart that schematically shows exemplary method steps of generating an optimized electronic neural network. As shown, method 100 includes creating a graph model and to identify modifiable levers in a given clinical scenario from identified relationships among sets of clinical data, community-based data, and molecular marker data to produce a set of identified modifiable levers in which the modifiable levers comprise clinical factors, medications, risk factors that influence outcomes, and/or interventions associated with a probability of a negative outcome (step 102). Method 100 also includes adjusting the set of identified modifiable levers using the electronic neural network to produce a reduction in a predicted probability of a negative outcome at a level of a given patient (step 104). FIG. 1B is a flow chart that schematically shows exemplary method steps of predicting and optimizing a medical outcome of a patient. As shown, method 200 includes identifying relationships among a set of characteristics of an initial patient population, a set of therapies, and a set of outcomes in a given clinical scenario to produce a set of identified relationships (step 202) and identifying modifiable levers that can be optimized in the set of identified relationships to produce a set of identified modifiable levers (step 204). In addition, method 200 also includes adjusting the set of identified modifiable levers applicable to a selected patient population to produce a reduction in a predicted probability of a negative outcome for the patient (step 206).

As a further illustration, FIG. 1C is a flow chart that schematically shows exemplary method steps according to some aspects disclosed herein. As shown, the method includes the use of a data management platform to create a data frame. The data management platform involves the use of mapping, predictive, and case selection algorithms to select features and outcome. Once the data frame is generated, a predictive imputation step is performed to predict missing data elements followed by the creation of a clustering graph model. The clustering graph model is inspected for modifiable risk factors which can become levers and an optimizing range is defined for each of the levers. The method also includes the use of a propagating graph neural network to simulate changes in network components and to predict the selected outcome. The process also produces an optimizing grid that is searched for a combination of levers that reduces the predicted risk of a selected outcome. As also shown, the optimizing grid is used for cluster-level optimization, clinical trial optimization, and training of optimizers.

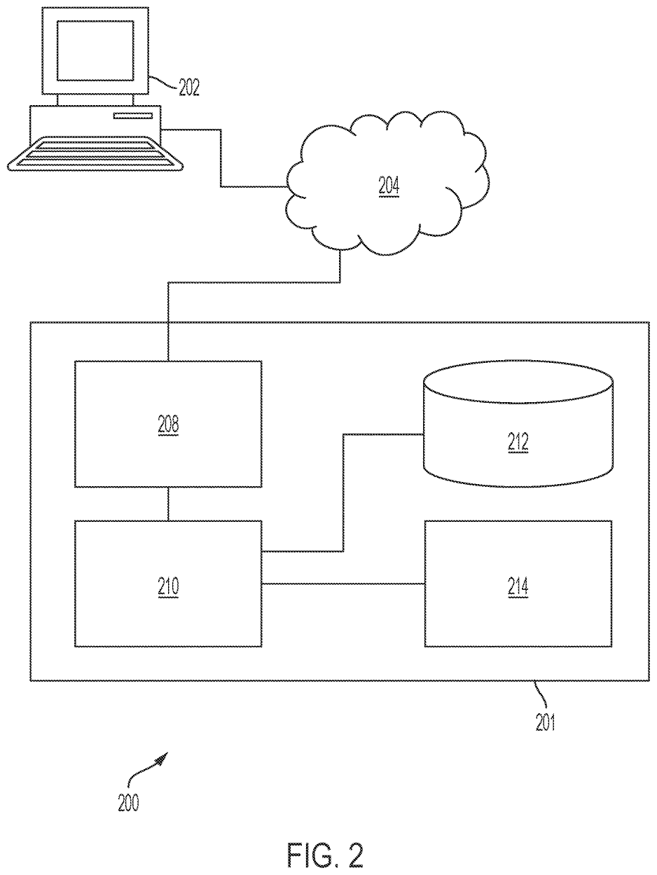

FIG. 2 is a schematic diagram of a hardware computer system 200 suitable for implementing various embodiments. For example, FIG. 2 illustrates various hardware, software, and other resources that can be used in implementations of any of methods disclosed herein, including method 100 and/or one or more instances of an electronic neural network. System 200 includes training corpus source 202 and computer 201. Training corpus source 202 and computer 201 may be communicatively coupled by way of one or more networks 204, e.g., the internet.

Training corpus source 202 may include an electronic clinical records system, such as an LIS, a database, a compendium of clinical data, or any other source of data suitable for use as a training corpus as disclosed herein. According to some embodiments, each component is implemented as a vector, such as a feature vector, that represents a respective tile. Thus, the term “component” refers to both a tile and a feature vector representing a tile.

Computer 201 may be implemented as any of a desktop computer, a laptop computer, can be incorporated in one or more servers, clusters, or other computers or hardware resources, or can be implemented using cloud-based resources. Computer 201 includes volatile memory 214 and persistent memory 212, the latter of which can store computer-readable instructions, that, when executed by electronic processor 210, configure computer 201 to perform any of the methods disclosed herein, including method 100, and/or form or store any electronic neural network, and/or perform any classification technique as described herein. Computer 201 further includes network interface 208, which communicatively couples computer 201 to training corpus source 202 via network 204. Other configurations of system 200, associated network connections, and other hardware, software, and service resources are possible.

Certain embodiments can be performed using a computer program or set of programs. The computer programs can exist in a variety of forms both active and inactive. For example, the computer programs can exist as software program(s) comprised of program instructions in source code, object code, executable code or other formats; firmware program(s), or hardware description language (HDL) files. Any of the above can be embodied on a transitory or non-transitory computer readable medium, which include storage devices and signals, in compressed or uncompressed form. Exemplary computer readable storage devices include conventional computer system RAM (random access memory), ROM (read-only memory), EPROM (erasable, programmable ROM), EEPROM (electrically erasable, programmable ROM), and magnetic or optical disks or tapes.

### II. Description of Example Embodiments

### Example 1: Determine Relationships Emerging From Integration Between Clinical, Community-Based, and Molecular Markers Using a Fully Connected Parsimonious Neural Network Approach

This example uses computational simulations to identify the levers (modifiable risk factors and interventions associated with the probability of negative outcomes) in a graph model and determines in silico whether optimization of those levers through a neural network at the individual patient level, results in a reduction in the predicted probability of negative outcomes. For example, a comprehensive network model using clinical, community-based, and molecular measures will improve prediction of early and sequential outcomes after hypoxic ischemic encephalopathy (HIE) and response to therapeutic hypothermia (TH) better than any single data source and optimization of the neural network at the patient-level using identified levers will result in a decreased probability of negative outcomes.

In this network-based approach, we used a subset of HIE clinical data collected to provide a simplified example and illustrate key concepts. As part of this study, we use the same analytic method below but with an expanded scope. This type of analysis involves four steps: 1) a clustering network to understand associations between various clinical factors and outcomes, 2) identifying potential clinical “levers” in the clustering network (“levers” are modifiable risk factors that influence outcomes either directly [adjacent nodes] or indirectly [connection possible only through one or more intermediary nodes]), 3) selection of an outcome and training a neural network to predict that outcome based on the inputs of the clustering network, and 4) simulating change in predictions when the clinical levers are activated and identify the combination of levers that minimizes the predicted risk of outcomes.

In this example, complete data were extracted for 182 HIE patients of whom 33(18 %) had an MRI score >1. We preselected 27 potential predictors based on clinical relevance, previous studies, and statistical associations with the MRI score, and each were manually assigned to a risk dimension, which describes the part of the clinical picture from which the risk factor emerged: maternal risk factors, birth circumstances, physiological status at birth, and postnatal complications. MRI score was discretized into two levels: normal (score=0) and abnormal (any other score is abnormal. In the analysis we use predictive imputation to handle missing variables (except for the primary prediction target which is not imputed resulting in the removal of those patients from the network), as we have successfully used this approach before.

For the study we are using the same clinical factors as previously described. A point-biserial correlation is calculated between each numeric feature and the binary indicator of abnormal MRI score. For categorical features, correlations with the outcome variable are assessed using the bias-corrected Cramer's V statistic. These correlations are used to identify trends and relationships between the various risk dimensions contributing to the abnormal MRI score and combine closely related factors into clusters (not all clusters necessarily have ≥1 variable). Clusters are then be combined into a network following previously presented methods. First, effect sizes and corresponding significance levels are calculated for each possible pair of features. For numeric-numeric pairs, a student's t-test are performed on Spearman's ρ, and the effect size is taken as ρ². A chi-squared test is implemented for categorical-categorical pairs, and the bias-corrected estimator of Cramer's V is used as the effect size parameter. In the case of mixed pairs, a Kruskal-Wallis analysis of variance test is performed with a Dunn's post hoc test, and the effect size is equal to Zₘₐₓ²/n (where Zₘₐₓ is the maximum absolute Z-score from the Dunn's test and n is the number of samples). Given that we are dealing with only a small number of features in this analysis, we have considered all the possible pairs of features to visualize any existing small effect relationship between them in a network. In other iterations, we expect a large number of features in the dataset, in that case our approach applies a false discovery rate correction method for multiple comparisons in the assessment of features relationship significance and filters non-significant feature pairs from the analysis to avoid overfitting. Second, a minimum-spanning tree (MST) is generated using the significant features and the calculated effect sizes. A method is used to assign each feature in the MST to a cluster in the network. In the MST, each node represents a feature, an edge between nodes indicates a significant relationship between features, edge width represents the strength of the relationship, node color denotes the risk dimension, and node size represents its centrality (i.e., number of adjacent nodes). The MST and the assigned clusters are then used to generate a cluster network, in which each node represents a cluster of features and an edge between nodes indicates a significant relationship between clusters. Clusters with no connection to any other cluster in the network are removed from the analysis. A simplified form of the network is then created to help in the selection of appropriate levers, where each cluster is given a title based on its most common risk dimension(s) in each cluster. The simplified network helps identify the critical dimensions of risk and of potential clinical levers.

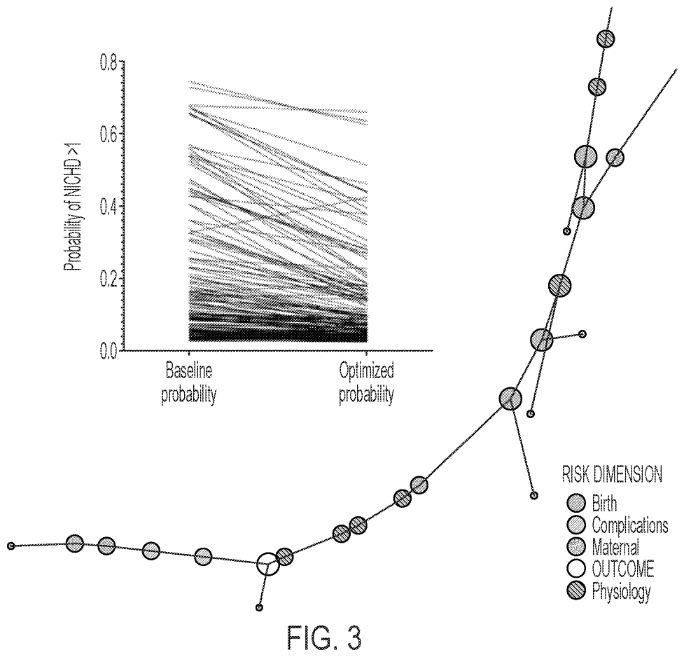

The cluster network generated in this example is depicted in FIG. 3. In this example, we selected three variables as potential levers: hematocrit (can be modified by red blood cell transfusions), platelet count (can be modified by platelet transfusions) and VIS in the early stages of TH. VIS in this context is a particularly good candidate for optimization, as there is a high level of practice variation, no set guidelines either in patient selection or intensity of treatment, and the intensity can be adjusted in a highly granular manner. In the architecture, we examine the cluster components to identify additional modifiable risk factors that can be used as levers to influence the probability of an outcome. Significant risk factors identified as part of the prediction models created can also be used to identify potential levers.

The third step is to create a prediction model for abnormal MRI score. Patients are first split into a balanced training dataset (75% of data) and a validation dataset (25% of data). The features in both the training and validation sets are standardized by removing the mean and scaling to unit variance. Next, a neural network architecture is defined with an input layer, one hidden layer, and an output layer. The input layer features a size equivalent to the number of features in the dataset. The number of neurons in the hidden layer is initially set equal to the total number of clusters from the MST. The weights of the neurons in the hidden layer are initialized with a uniform Glorot initializer and are optimized through training of the neural network. The size of the hidden layer is set as a tunable parameter. The hidden layer uses the hyperbolic tangent activation function, and a dropout of 60% is applied. The output layer features one neuron and is assigned a sigmoid activation function. The model is compiled with a binary cross entropy loss function, as well as an ADADELTA optimization algorithm. The two tunable hyperparameters (i.e. hidden layer size and number of epochs) are optimized within set boundaries using 5-fold cross-validation area under the receiver operating characteristic curve (AUC) and Bayesian optimization. This optimization process is performed in using the training dataset and results in a single set of hyperparameters that optimizes the mean cross-validation AUC. Using this set of hyperparameters, the neural network is then trained with the training dataset and evaluated on its ability to predict the target variable in that dataset. In our example, the actual patient dataset was fed into the neural network initially to train the network and evaluated in predicting the probability of the target variable. Our neural network achieved an AUC of 0.834, which is not surprising given the limited number of features we chose to include, more comprehensive networks have a higher degree of accuracy.

The final step is to simulate the effect of the levers on outcome probability and identify the combination of levers most likely to reduce this probability. In this simulation, a data grid was tailored to include the three modifiable risk factors (hematocrit (HCT), platelet count (PLT) and vasoactive inotropic support (VIS)) and vary each of them in a defined clinically relevant range. The data grid included all the possible combinations of the modifiable risk factors within the defined ranges. A synthetic dataset is formed by repeating this data grid for each patient. Therefore, the synthetic dataset has all the 27 features filled with grid values for the HCT, PLT and VIS and values for the remaining non-modifiable predictors are filled with the actual patient data. The synthetic dataset is then fed into the trained neural network and the probability of outcome for a given combination of levers is calculated for each patient. Examination of the data grid identifies lever combinations with the lowest outcome probability, and corresponding modifiable risk factors values.

In this example we found that the overall probability of MRI score >1 was 6.2% lower (absolute difference) in the optimized dataset (11.8%) than in the original dataset (18.0%). In this cohort of 182 patients, should this reduction in predicted risk fully translate into clinical outcomes, ~11/33 adverse outcomes could have been avoided.

The optimizing network disclosed herein can be used clinically by identifying patients who are more/less likely to benefit from each therapeutic approach, including but not limited to TH and adjunct therapies; establishing distinct phenotypes that are more/less likely to benefit from specific therapeutic approaches thus providing the impetus and pilot data for stratified clinical trials; and informing agenda for the search and discovery of new molecular therapeutic targets by focusing on risk clusters which do not include modifiable risk factors.

### Example 2: Method of Outputting Levers, Predicted Data, and Probability of Outcomes, and Calculating Benefit

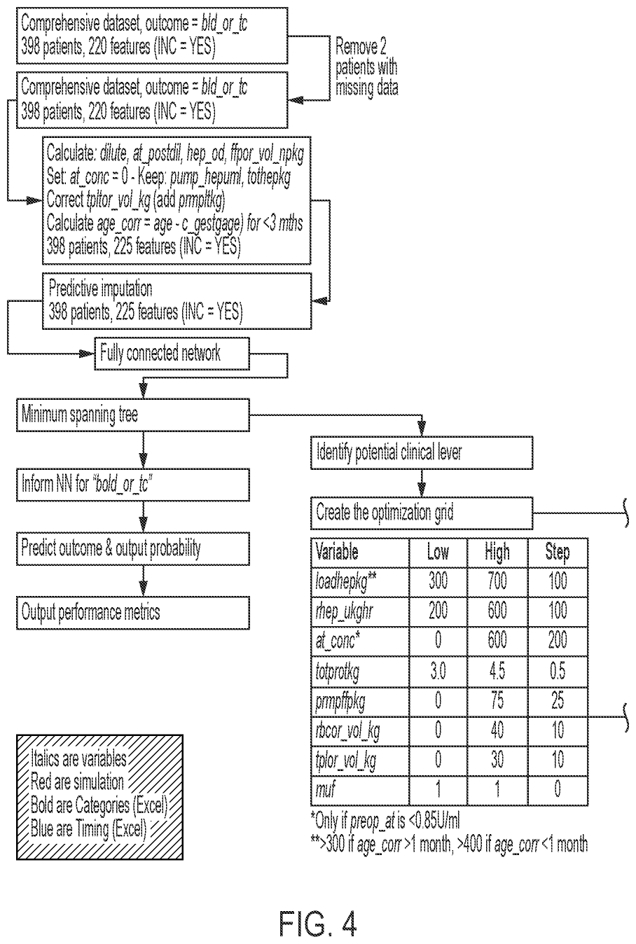

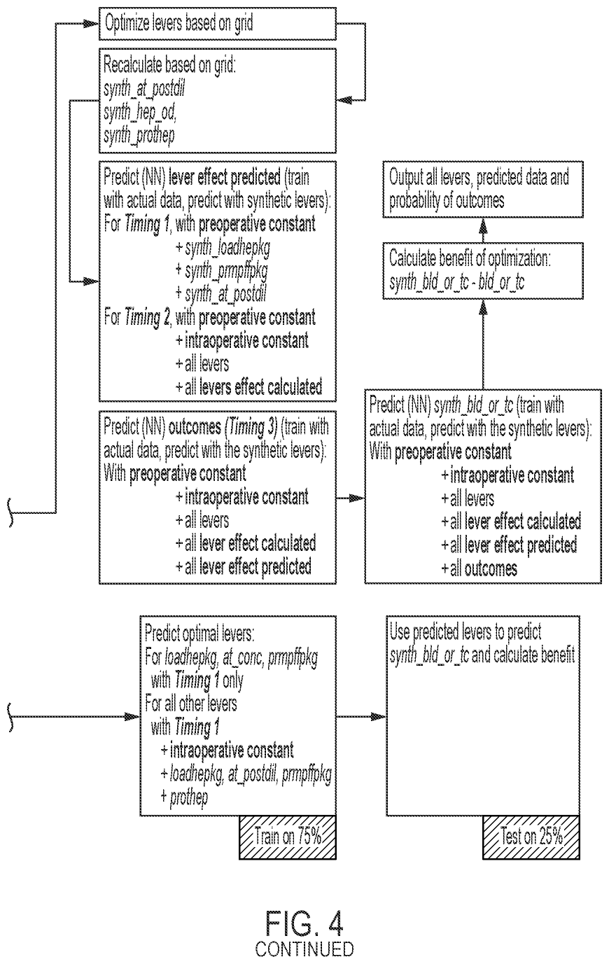

FIG. 4 is a flow chart that schematically shows exemplary method steps according to some aspects disclosed herein. Specifically, this figure overlays the steps previously described with the specific parameters (including patient population, variables, modifiable risk factors, propagation, optimization grid and prediction of optimal parameters at the patient level) from a second pilot project using this technology.

While the invention has been described with reference to the exemplary embodiments thereof, those skilled in the art will be able to make various modifications to the described embodiments without departing from the true spirit and scope. The terms and descriptions used herein are set forth by way of illustration only and are not meant as limitations. In particular, although the method has been described by examples, the steps of the method can be performed in a different order than illustrated or simultaneously. Those skilled in the art will recognize that these and other variations are possible within the spirit and scope as defined in the following claims and their equivalents.

1. (canceled)

2. A computer-implemented method of predicting and optimizing a medical outcome of a patient, the method comprising:
identifying one or more relationships among a set of characteristics of an initial patient population, a set of therapies, and a set of outcomes in a given clinical scenario to produce a set of identified relationships;
identifying one or more modifiable levers that can be optimized in the set of identified relationships to produce a set of identified modifiable levers; and,
adjusting the set of identified modifiable levers applicable to a selected patient population to produce a reduction in a predicted probability of a negative outcome for the patient, thereby predicting and optimizing the medical outcome of the patient.

3. (canceled)

4. The method of claim 2, wherein the medical outcome comprises a therapeutic outcome.

5. The method of claim 2, wherein the modifiable levers comprise clinical factors, medications, risk factors that influence outcomes, and/or interventions associated with a probability of a negative outcome.

6. The method of claim 2, wherein the given clinical scenario comprises thrombohemorrhagic complications of pediatric cardiac bypass, hypoxic ischemic encephalopathy (HIE), or a response to therapeutic hypothermia (TH).

7. The method of claim 2, further comprising determining an optimal value for each of one or more levers in a set of optimized levers for the patient to produce a set of optimal values for the patient.

8. The method of claim 7, further comprising using the set of optimal values for the patient to identify one or more therapies for the patient to produce a set of identified therapies.

9. The method of claim 8, further comprising administering one or more identified therapies in the set of identified therapies to the patient.

10. The method of claim 2, further comprising using one or more selected therapies as levers in the selected patient population.

11. The method of claim 10, wherein the selected therapies comprise a pharmaceutical therapy.

12. The method of claim 10, further comprising predicting one or more effects, and/or one or more factors associated with a positive and negative effect, of the selected therapies to produce a set of predicted effects and/or factors.

13. The method of claim 12, further comprising using the set of predicted effects and/or factors to design of a clinical trial related to at least one of the selected therapies.

14. The method of claim 13, wherein the clinical trial relates to a clinical scenario that comprises thrombohemorrhagic complications of pediatric cardiac bypass.

15-21. (canceled)

22. The method of claim 2, comprising predicting missing data elements in a given data set.

23. The method of claim 22, comprising predicting the missing data elements using a variable-specific self-tuning, single-layer electronic neural network.

24. The method of claim 2, comprising predicting missing variables for a given patient in the initial patient population and/or the selected patient population based at least in part on other data present in the given data set to produce a patient-level prediction for the given patient.

25-28. (canceled)

29. The method of claim 2, comprising simulating an effect of lever optimization on downstream variables and outcomes by (a) converting a minimum spanning tree to a clustering electronic neural network comprising a layer constrained by a structure of the minimum spanning tree, (b) organizing one or more features from the minimum spanning tree sequentially based on biological and/or temporal ordering and predicting one or more variables that are downstream of the modifiable levers, and (c) predicting a probability of a prespecified outcome for a given patient in the initial patient population and/or the selected patient population using initial or current values of the modifiable levers to produce one or more benchmark values that are used in adjusting the set of identified modifiable levers applicable to the selected patient population.

30. (canceled)

31. The method of claim 30, comprising producing an optimization grid for the given patient, wherein the levers in the set of identified modifiable levers are allowed to vary based on one or more pre-defined ranges and steps.

32. The method of claim 31, wherein the optimization grid comprises substantially all combinations of the levers in the set of identified modifiable levers.

33-40. (canceled)

41. A system for predicting and optimizing a medical outcome of a patient, the system comprising:
a processor; and
a memory communicatively coupled to the processor, the memory storing instructions which, when executed on the processor, perform operations comprising:
identifying one or more relationships among a set of characteristics of an initial patient population, a set of therapies, and a set of outcomes in a given clinical scenario to produce a set of identified relationships;
identifying one or more modifiable levers that can be optimized in the set of identified relationships to produce a set of identified modifiable levers; and,
adjusting the set of identified modifiable levers applicable to a selected patient population to produce a reduction in a predicted probability of a negative outcome for the patient.

42. (canceled)

43. A computer readable media comprising non-transitory computer executable instruction which, when executed by at least electronic processor perform at least:
identifying one or more relationships among a set of characteristics of an initial patient population, a set of therapies, and a set of outcomes in a given clinical scenario to produce a set of identified relationships;
identifying one or more modifiable levers that can be optimized in the set of identified relationships to produce a set of identified modifiable levers; and,
adjusting the set of identified modifiable levers applicable to a selected patient population to produce a reduction in a predicted probability of a negative outcome for the patient.
